(19) United States
(12) Patent Application Publication
(10) Pub. No.: US 2026/0034332 A1
(43) Pub. Date: Feb. 5, 2026

(54) Customizable Pressure Therapy Autism Helmet

(71) Applicant: Arthur David Stanton, Garland, TX (US)

(72) Inventor: Arthur David Stanton, Garland, TX (US)

(21) Appl. No.: 18/793,832

(22) Filed: Aug. 4, 2024

(51) Int. Cl. A61M 21/02; A42B 3/06; A61H 9/00; A61M 21/00

(52) U.S. Cl. CPC A61M 21/02; A42B 3/06; A61H 9/0078; A61H 2201/10; A61H 2201/1246; A61H 2201/1253; A61H 2201/1607; A61H 2201/5058; A61H 2201/5082; A61H 2201/5097; A61H 2205/021; A61H 2230/065; A61H 2230/505; A61M 2021/0027; A61M 2021/0044; A61M 2021/0066; A61M 2205/0205; A61M 2205/3368; A61M 2205/3584; A61M 2205/36; A61M 2230/06; A61M 2230/50

(57) ABSTRACT

A Customizable Pressure Therapy Autism Helmet provides personalized pressure therapy for children with autism. It features an air compressor that inflates baffles to apply soothing pressure, controlled via an iPhone. The helmet monitors pulse rate, body temperature, voice patterns, and motion to detect stress and activity levels. It also tracks sleep quality, including duration and stages, and offers additional features like soothing sounds, music playback, and communication between mother and child. A QR code system allows teachers to monitor the child in a classroom setting. Manual pressure activation is available via a button. This device aims to preemptively calm autistic children through personalized pressure and audio stimulation.

### FIELD OF THE INVENTION

This invention pertains to a therapeutic helmet specifically designed for individuals with autism, providing customizable pressure therapy and integrated sensory features to aid in managing and mitigating mood swings and anxiety.

### BENEFITS OF THE INVENTION

The Customizable Pressure Therapy Autism Helmet offers numerous benefits for children with autism. It detects mood swings before the autistic individual becomes unmanageable, significantly reducing or stopping severe outbursts.

This is accomplished by monitoring vital signs and logging effective calming actions described infra. The helmet tailors interventions to each child's needs. It also tracks sleep patterns and initiates preemptive actions to help the child sleep better and longer. Vital information can be logged and transmitted to teachers, allowing continuous monitoring and support in educational settings. The helmet provides significant benefits in situations where parents cannot immediately address issues, such as during traffic; it intervenes and calms the child autonomously.

Unlike other monitoring systems that only alert to situations, this helmet administers solutions before the behaviors escalate out of control. Its ability to provide early detection, tailored interventions, and comprehensive monitoring greatly enhances the child's well-being and quality of life.

### BACKGROUND OF THE INVENTION

Autistic children often struggle with controlling their emotions and understanding social situations, which can lead to elevated and sometimes violent behaviors from seemingly small issues. In most cases, caregivers are not aware of the building anxiety and pressure within the child until the mood swing has escalated to an uncontrollable level, manifesting through verbal outbursts or physical actions.

The Customizable Pressure Therapy Autism Helmet aims to detect this building anxiety and administer preemptive actions that can either lessen the severity of the mood swings or completely prevent them. Current methods of managing these challenges are only employed after the child has already lost control. The caregiver or user of the device deploys intervention methods typically involving calm talking, attempting to distract the child with happier situations, and applying pressure treatments such as compressing the child's head or using heavy blankets.

The Customizable Pressure Therapy Autism Helmet provides a unique and effective solution by utilizing advanced monitoring systems to detect early signs of anxiety and mood swings. It monitors the child's temperature and can direct cool air down the back of the neck for a calming effect. The helmet also monitors the pulse rate and activity sensors to determine erratic behaviors and speech fluctuations to assess stress levels. The helmet features an advanced monitoring system that includes two strategically placed microphones on each side near the mouth of the child. These microphones continuously record the child's voice, analyzing key vocal parameters such as tone, volume, and stress levels. This auditory data is crucial for determining the child's emotional state, differentiating between stress and happiness. In addition to the helmet's capabilities, the child will wear a wristband that houses various sensors. This wristband is equipped with advanced sensors to collect vital physiological data, such as heart rate, body temperature, and movement. By relocating the sensors to the wristband, the design ensures that the helmet remains lightweight and comfortable for the child. The wristband continuously transmits the collected data to the helmet, allowing for a comprehensive assessment of the child's physiological state. By integrating the auditory data from the microphones with the physiological data from the wristband, the system provides a comprehensive assessment of the child's overall mood and well-being. This holistic approach enhances the ability to monitor the child's well-being accurately and provides timely interventions as needed, ensuring the child's comfort and safety. By logging actions such as playing specific songs or white noise, the helmet can monitor vital signs and determine which interventions work best for each individual child. This capability does not exist in any current methods or devices, making this helmet an innovative approach to managing autism-related challenges.

The Customizable Pressure Therapy Autism Helmet provides a unique and effective solution by utilizing advanced monitoring systems to detect early signs of anxiety and mood swings. It monitors the child's temperature and can direct cool air down the back of the neck for a calming effect. The helmet also monitors the pulse rate and activity sensors to determine erratic behaviors and speech fluctuations to assess stress levels. By logging actions such as playing specific songs or white noise, the helmet can monitor vital signs and determine which interventions work best for each individual child. This capability does not exist in any current methods or devices, making this helmet an innovative approach to managing autism-related challenges.

Autistic children can be incredibly loving and gentle, but when a mood swing occurs, it can result in extreme violence, including screaming, scratching, kicking, and self-destructive actions like head-banging or pounding fists on walls and sometimes even others around them. Preventing or reducing the severity of these actions is crucial for the child's well-being and helps teach appropriate behavior while reducing stress for caregivers.

Feedback from caregivers and professionals has been overwhelmingly positive, with many expressing amazement and excitement with this new device. They have often stated that once a mood swing has been escalated, it is nearly impossible to contain. The ability to administer preemptive and automatic interventions is seen as a groundbreaking advancement, offering hope for better management of autistic behaviors and improved quality of life for both children and their caregivers.

In summary, the Customizable Pressure Therapy Autism Helmet addresses a significant unmet need by providing early detection and intervention for mood swings in autistic children, a capability that current methods lack. This innovative device offers a proactive and effective solution, greatly benefiting both the children and their caregivers.

### SUMMARY OF THE INVENTION

The therapeutic helmet for children with autism is an innovative device designed to manage and mitigate the intense mood swings often experienced by these children. The helmet integrates several advanced features that work together to provide a cohesive and comprehensive solution for early detection and intervention, ensuring the child's safety and comfort while aiding the caregiver in managing potentially disruptive episodes.

The helmet monitors the children and collects data to determine which actions work best, tailoring interventions to each child's needs. Its advanced features and integrated technologies provide early detection, proactive intervention, and continuous support, ensuring better outcomes for both the child and the caregiver.

### DETAILED DESCRIPTION

### Detailed Description—Air Baffles

The air baffles within the therapeutic helmet are crafted from a robust, child-safe plastic, similar to the materials used in children's life vests and swimming pool rafts. This choice ensures durability and flexibility, allowing the baffles to withstand repeated inflation and deflation cycles without losing their structural integrity. To enhance comfort, the side of the baffles that contacts the child's head is coated with a soft, breathable fabric, such as a velvet-like material. This coating prevents the child's head from becoming hot or sweaty, ensuring a comfortable and soothing experience.

The air compression system responsible for inflating the baffles offers multiple options to ensure versatility and reliability. One possible mechanism is a diaphragm-type compressor. Diaphragm compressors are known for their efficiency and quiet operation, making them suitable for a therapeutic device. These compressors use a flexible membrane to compress air, providing a gentle and consistent inflation of the baffles. Another viable option is a piston-driven compressor.

Piston compressors are renowned for their robust performance and ability to generate higher pressures. They work by using a piston to compress air within a cylinder, which allows for precise control over the pressure levels. This type of compressor is particularly useful when adjustable and consistent pressure is required for therapeutic purposes. Alternatively, the helmet could incorporate a compressed CO2 system. This system utilizes small, rechargeable CO2 canisters to inflate the baffles.

The advantages of a CO2 system include rapid inflation and a compact design, making it easy to integrate into the helmet. The canisters can be easily replaced or recharged, ensuring continuous operation without the need for an external power source. In addition to these automatic inflation methods, the helmet may also feature a manual pump. The manual pump allows for the inflation of a small holding tank, providing an additional layer of functionality. This ensures that the helmet can still be used even in the absence of an electric or CO2-based compressor.

The manual pump is designed to be user-friendly, allowing caregivers or the child to inflate the baffles as needed with minimal effort. The integration of these various air compression methods ensures that the helmet can provide consistent and adjustable pressure tailored to the individual needs of the child. The ability to control the intensity and duration of the pressure is crucial for the therapeutic effectiveness of the device. By offering multiple options for air compression, the helmet can accommodate different preferences and situations, ensuring that the child receives the most effective and soothing therapy possible. All components, including the air compressor, air baffles, and manual pump, are securely integrated into the helmet's structure.

This integration ensures that the device remains compact and unobtrusive, allowing the child to wear it comfortably for extended periods. The materials used for the air baffles and the compressor system are all hypoallergenic and safe for sensitive skin, further enhancing the device's suitability for children with autism. The careful design and selection of materials for the air baffles and the air compression system highlight the innovative approach taken to address the unique needs of children with autism. By focusing on providing reliable, adjustable, and comfortable pressure therapy, the therapeutic helmet aims to enhance the well-being of these children through personalized therapeutic techniques.

In summary, the therapeutic helmet's air baffles and compression system are meticulously designed to offer reliable and adjustable pressure therapy. The use of high-quality materials and the incorporation of multiple inflation methods ensure that the helmet delivers consistent and effective therapy. The attention to detail in the design and functionality of the air baffles and compressor system underscores the commitment to providing a soothing and beneficial experience for children with autism.

### Detailed Description—App Features

Autistic children can experience sudden and severe mood swings, which can be verbally or physically violent and challenging to manage. These outbursts can occur anywhere, such as in a mall or while driving in heavy traffic, creating stressful situations for both the child and the caregiver. The therapeutic helmet addresses this by offering remote control features designed to help manage and mitigate these mood swings effectively.

The primary method for controlling the helmet is through an iPhone app, which acts as the main interface. This app is designed to function on multiple levels, providing comprehensive control over the helmet's features and ensuring that the parent, teacher, or any authorized person can manage the child's needs effectively. The methods for this monitoring are by considering the temperature, pulse rate, activity sensors, and voice patterns.

An increasing pulse rate, rising temperature, stress levels detected in the child's voice, and erratic activity can be indicative of an impending mood swing. The app is designed to sense these changes and, based on previously gathered data, initiate appropriate actions to prevent or mitigate the mood swing. The therapeutic helmet features an advanced monitoring system that includes two strategically placed microphones on each side near the mouth of the child. These microphones continuously record the child's voice, analyzing key vocal parameters such as tone, volume, and stress levels. This auditory data is crucial for determining the child's emotional state, differentiating between stress and happiness. In addition to the helmet's capabilities, the child will wear a wristband that houses various sensors. This wristband is equipped with advanced sensors to collect vital physiological data, such as heart rate, body temperature, and movement. By relocating the sensors to the wristband, the design ensures that the helmet remains lightweight and comfortable for the child. The wristband continuously transmits the collected data to the helmet, allowing for a comprehensive assessment of the child's physiological state. By integrating the auditory data from the microphones with the physiological data from the wristband, the system provides a comprehensive assessment of the child's overall mood and well-being. This holistic approach enhances the ability to monitor the child's well-being accurately and provides timely interventions as needed, ensuring the child's comfort and safety. For instance, if a slight increase in pulse rate is detected, the app might trigger a low-pressure inflation of 5 pounds to the helmet, which can help soothe the child. In other situations, the elevated pulse might suggest the need for auditory interventions, such as playing low tones through the helmet, which have a calming effect.

The app also allows for the playback of recorded messages from the parent. These messages can be soothing and relaxing words, pre-recorded specifically to help calm the child during moments of distress. In more severe situations, higher pressure levels can be applied, based on the child's past responses to such interventions. These settings are predetermined by the parent, leveraging past experiences to ensure that the child receives the most effective response in any given situation. In scenarios such as a car ride or a visit to the mall, the auto mode can be particularly useful.

This mode allows the app to operate independently, continuously monitoring the child's pulse rate, temperature, activity sensors, and voice patterns and taking pre-programmed actions as necessary. Even when the function is not set to auto mode, the app still collects and logs pulse rate, temperature, activity and voice patterns. This data is used to build a detailed profile of the child, which helps in understanding which actions are most effective in various situations.

Another important feature is the notification system. If the app detects signs of an oncoming mood swing, it can send an alert to the parent's iPhone. This early warning system allows the parent to take immediate action, whether it be initiating a calming pressure sequence or playing a soothing audio message. These notifications ensure that the parent is always aware of the child's state and can intervene before the mood swing escalates.

The app also supports pre-recorded voice messages from the parent. Some children respond well to familiar, calming voices, and the ability to play these messages can be crucial in preventing or mitigating a mood swing. Additionally, favorite songs or specific low and soothing tones can be uploaded to the app and played when needed. This customization ensures that the interventions are tailored to the child's unique preferences and needs.

Furthermore, the app allows for the inclusion of additional authorized users, such as another parent or a teacher. For instance, a teacher could have a master app capable of monitoring multiple autistic children simultaneously. If a teacher has a classroom with several students, the parent can generate a QR code that the teacher scans to gain access to the child's profile. This profile includes all relevant data and preset actions that the child responds to positively.

The teacher can then monitor the child's pulse rate, temperature, activity sensors, and voice patterns in real-time and take appropriate actions as needed. In the classroom setting, if the app alerts the teacher to signs of anxiety in a child, the teacher can immediately initiate the pre-set actions based on the child's profile. Additionally, by pressing and holding the child's picture on the teacher's app interface, the teacher can communicate directly with the child through the helmet, providing reassurance and guidance.

The most crucial feature of the helmet is its ability to monitor the child's activities continuously and compile a comprehensive data profile. This profile enables the system to anticipate potential mood swings and take preemptive actions to control or dissipate them. The ability to predict and respond to the child's needs in real-time helps in managing mood swings more effectively, reducing the need for physical restraint or other disruptive interventions. In conclusion, the remote control features of the therapeutic helmet are designed to provide a comprehensive, user-friendly, and effective means of managing and mitigating mood swings in autistic children.

By leveraging advanced technology and data-driven insights, the helmet helps caregivers and teachers to create a safer, more controlled environment for the child. This not only enhances the child's well-being but also provides peace of mind to those responsible for their care.

### Detailed Description—Pulse Rate Monitoring System

The pulse rate monitoring system integrated into the therapeutic helmet for children with autism leverages advanced sensor technology and communication protocols. This system is designed to continuously monitor the child's pulse rate, temperature, activity, and voice patterns, providing real-time data to help anticipate and manage mood swings effectively. The helmet features an advanced monitoring system that includes two strategically placed microphones on each side near the mouth of the child. These microphones continuously record the child's voice, analyzing key vocal parameters such as tone, volume, and stress levels. This auditory data is crucial for determining the child's emotional state, differentiating between stress and happiness.

### Detailed Description—Sensor Technology

The sensors and monitoring devices are strategically placed either within the helmet or on a wristband worn by the child. Both sets of devices can track the child's pulse rate, temperature, activity levels, and voice patterns. By relocating the sensors to the wristband, the design ensures that the helmet remains lightweight and comfortable for the child. The wristband continuously transmits the collected data to the helmet, allowing for a comprehensive assessment of the child's physiological state.

By integrating the auditory data from the microphones with the physiological data from the wristband, the system provides a comprehensive assessment of the child's overall mood and well-being. This holistic approach enhances the ability to monitor the child's well-being accurately and provides timely interventions as needed, ensuring the child's comfort and safety.

### Detailed Description—Data Collection and Initial Processing

The sensors continuously collect pulse rate data, which is transmitted to the helmet's onboard processor. This processor is designed to handle real-time data analysis, filtering out noise and irrelevant signals to ensure accuracy. The processor performs initial data processing, identifying significant changes or trends in the pulse rate that might indicate the onset of a mood swing.

Detailed Description—Wireless Communication with the App

The helmet employs Bluetooth technology to wirelessly transmit the processed data to the accompanying iPhone app. The use of Bluetooth ensures low power consumption and reliable data transmission. The app, acting as the central control hub, receives this data in real-time, allowing for continuous monitoring of the child's physiological and behavioral indicators.

### Detailed Description—Real-Time Monitoring and Analysis

The iPhone app is equipped with advanced algorithms designed to analyze the incoming pulse rate, temperature, motion and voice pattern data. By continuously comparing current data rates with baseline readings and historical data, the app can detect significant deviations that may signal an impending mood swing. These algorithms are customizable, allowing parents to adjust sensitivity levels based on their child's unique patterns and needs.

### Detailed Description—Automatic Interventions

When the app detects a variation with pulse rate, temperature, activity or voice pattern data indicative of a potential mood swing, it cross-references this data with the child's personalized profile. This profile is built from historical data and includes information about the child's past responses to various interventions. Based on this analysis, the app can automatically initiate specific actions to help soothe the child. These actions may include:

Adjusting Air Baffles: The app can trigger the helmet's air compression system to inflate the air baffles at a pre-set pressure level. For instance, a slight increase in pulse rate might result in a gentle 5-pound pressure application, which can help calm the child.

Playing Soothing Tones: The app can play low-frequency tones through the helmet's integrated speakers, designed to provide auditory comfort.

Delivering Voice Messages: The app can play pre-recorded voice messages from the parent, offering comforting and familiar words to the child.

### Detailed Description—Manual Interventions

In addition to automatic responses, the app provides parents and caregivers with the ability to manually intervene when necessary. If the child's pulse rate data triggers an alert, the parent can review the situation and decide on the appropriate action. The app's interface allows for easy control over all helmet functions, enabling the caregiver to manually adjust air pressure, play specific audio tracks, or communicate directly with the child through the helmet's audio system.

### Detailed Description—Alerts and Notifications

The app is designed to send real-time alerts and notifications to the parent or caregiver's iPhone whenever the sensors exceeds a predefined threshold. These notifications ensure that the caregiver is immediately aware of potential mood swings and can take prompt action. Alerts can be customized based on the child's profile, ensuring that they are only triggered by significant deviations to avoid unnecessary interruptions.

### Detailed Description—Data Logging and Profile Building

All data collected by the helmet is logged and stored within the app. This historical data is crucial for building a comprehensive profile of the child's physiological responses. Over time, the app identifies trends and patterns in the pulse rate, temperature, activity sensors, and voice patterns, which helps in refining the child's profile. For example, the app might determine that a certain pressure level or audio tone is particularly effective at reducing pulse rates during high-stress moments.

### Detailed Description—Insights and Reports

The app can generate detailed reports and insights based on the collected data, providing valuable feedback to parents and caregivers. These reports can include:

Trends: Graphical representations of the child's pulse rate, temperature, activity sensors, and voice patterns over time, highlighting periods of increased stress or relaxation.

Intervention Effectiveness: Analysis of how different interventions impact the child's physiological and behavioral indicators, helping to identify the most effective strategies.

Customized Recommendations: Based on the data, the app can suggest adjustments to the intervention protocols to enhance their effectiveness.

Detailed Description—Integration with Other Features

The pulse rate, temperature, activity sensors, and voice pattern monitoring system is seamlessly integrated with other helmet features, ensuring a holistic approach to managing the child's well-being. For instance, if the data indicates an impending mood swing, the app can simultaneously adjust the air baffles, play soothing tones, and alert the parent. This integrated approach ensures that the child receives comprehensive support tailored to their immediate needs.

### Detailed Description—Secure Data Handling

Data security and privacy are paramount. All data transmitted between the helmet and the app is encrypted to prevent unauthorized access. The app also allows parents to control data sharing settings, ensuring that sensitive information is only accessible to authorized individuals.

### Detailed Description—Customization and Adaptability

The monitoring system and the app are highly customizable, allowing parents to tailor the settings to their child's specific needs. Sensitivity levels, intervention thresholds, and response protocols can all be adjusted to ensure the most effective support. This adaptability is crucial for accommodating the unique and evolving needs of each child.

### Detailed Description—Sensor Inputs

The helmet can track the child's physical activity levels throughout the day including pulse rate, temperature, activity sensors, and voice patterns. This information helps in understanding how all the different areas correlate with mood swings and stress levels, providing a more comprehensive picture of the child's overall well-being.

### Detailed Description—Sleep Tracking

The helmet and wrist band is equipped to monitor the child's sleep patterns, including duration and quality of sleep. This feature is particularly useful for identifying how sleep disturbances might contribute to mood swings. The app can provide insights into sleep quality, helping caregivers to implement better sleep routines and identify potential issues.

### Detailed Description—Sleep Quality Analysis and Intervention

The helmet's sensors can also analyze sleep quality by monitoring sleep stages (light, deep, and REM sleep) including sleep duration. If the helmet detects signs of discomfort or restlessness during the night, it can initiate calming interventions such as gentle pressure adjustments or playing soothing audio. These features help ensure that the child experiences a more restful and restorative sleep.

### Detailed Description—QR Code Access Feature

The therapeutic helmet includes a QR code access feature to facilitate easy and secure sharing of the child's profile and control settings with authorized individuals such as teachers or caregivers. This feature ensures that multiple caregivers can monitor and manage the child's needs effectively.

### Detailed Description—Qr Code Generation

The QR code is generated through the iPhone app associated with the therapeutic helmet. The parent or primary caregiver can create a unique QR code that encodes essential data about the child's profile, including:

Personal Information: Basic details about the child, such as their name and age.

Intervention Protocols: Pre-set responses for different thresholds, including pressure adjustments, audio settings, cooling and voice messages.

Activity and Sleep Data: Historical data on the child's activity levels and sleep patterns, helping new caregivers understand the child's typical behavior and needs.

The QR code is generated within the app and can be displayed on the screen for easy scanning or printed out for physical use.

### Detailed Description—Granting Access to Caregivers

To grant access, the parent provides the QR code to the teacher or other caregiver. The authorized individual scans the QR code using their own device, which has the companion app installed. This scanning process securely transfers the encoded data to the caregiver's app, allowing them to access the child's profile and manage the helmet's functions.

Detailed Description—Integration with App and Helmet Functions
Integration with App and Helmet Functions

Once the QR code is scanned and the data is transferred, the caregiver's app synchronizes with the therapeutic helmet. This synchronization includes:

Profile Synchronization: The child's profile, including personalized intervention protocols, is loaded onto the caregiver's app.

Real-Time Monitoring: The caregiver can now monitor the child's pulse rate, temperature, activity sensors, and voice patterns in real-time, just like the primary caregiver.

Control Features: The caregiver gains control over the helmet's features, such as adjusting air pressure in the baffles, playing soothing tones, and delivering pre-recorded voice messages. These controls are available based on the permissions set by the primary caregiver.

### Detailed Description—Ensuring Security and Privacy

Security and privacy are paramount in the QR code access feature. The following measures are implemented:

Encrypted Data: The data encoded in the QR code is encrypted to prevent unauthorized access and ensure that only the intended caregiver can use it.

Access Permissions: The primary caregiver can set specific permissions for each QR code, limiting the scope of access (e.g., only allowing monitoring without control capabilities).

Revocable Access: If needed, the primary caregiver can revoke access by deactivating the QR code through the app, ensuring that the caregiver no longer has access to the child's profile and control features.

### Detailed Description—Use Case in Educational Settings

In a classroom setting, a teacher with access to multiple children's profiles can use the QR code system to manage several helmets simultaneously. Here's how it works:

Multiple Profiles: The teacher's app can store multiple children's profiles, each accessed via a unique QR code provided by the respective parents.

Real-Time Alerts: The app alerts the teacher if any child's physiological and behavioral indicators indicate an impending mood swing, allowing the teacher to take immediate action.

Direct Communication: The teacher can communicate directly with the child through the helmet's audio system by selecting the child's profile and activating the voice message feature.

### Detailed Description—Simplified Management and Monitoring

The QR code access feature simplifies the management and monitoring process for caregivers by providing an efficient and secure method to share the child's therapeutic settings. This system ensures that all caregivers have the necessary information and tools to provide consistent and effective support to the child, whether at home, in school, or in other environments.

The QR code access feature enhances the usability and flexibility of the therapeutic helmet by allowing multiple caregivers to manage and monitor the child's needs effectively. By leveraging QR technology, the system ensures secure and efficient data sharing, real-time monitoring, and comprehensive control over the helmet's functions. This innovative approach facilitates a collaborative care environment, ensuring that children with autism receive the consistent and personalized support they need across different settings.

### Detailed Description—Autistic Helmet Child Controls

### Manual Activation Feature for the Therapeutic Helmet

The manual activation feature of the therapeutic helmet is designed to provide children with a limited, controlled ability to interact with their helmet. This feature is carefully integrated to ensure the child's safety and maximize the therapeutic benefits while maintaining caregiver control over the primary functions.

### Limited Access for Safety

Children have limited access to the helmet's manual controls, and this access is granted and regulated by the caregiver through the app. This ensures that the child can only use the manual features in a manner that is safe and beneficial.

### Manual Pressure Control

On the left side of the helmet, there is a button specifically designed for the child to manually control the inflation of the air baffles. When the child presses this button, the air compressor is activated, inflating the baffles to a pre-set pressure level determined by the caregiver. This level is optimized based on historical data gathered from the child's previous sessions, ensuring that the pressure applied is both safe and effective in providing the desired calming effect.

### Air Pressure Release

On the left side of the helmet just below the inflation button, there is a button dedicated to releasing the air pressure. This button allows the child to deflate the air baffles manually. The placement of this button on the opposite side of the inflation button minimizes the risk of accidental activation. This release function gives the child a sense of control and autonomy, which can be particularly soothing during moments of stress.

### Detailed Description—Audio Control

Adjacent to the air pressure release button on the left side is another button that controls the helmet's audio features. This button is designed to cycle through different audio options pre-set by the caregiver. The audio options include favorite songs and soothing tones, which have been determined to provide the best benefit based on data gathered from the child's past experiences.

First Press: When the child presses this button once, a favorite song plays through the helmet's integrated speakers. This song is chosen by the caregiver and is typically one that has a calming effect on the child.

Second Press: Pressing the button again will switch to a different song, also selected by the caregiver based on what has been effective in soothing the child.

Third Press: A third press will activate a calming tone, which can provide auditory comfort to the child. These tones are pre-selected and configured by the caregiver to ensure they are effective.

Detailed Description—Integration with Other Features

The manual activation feature is seamlessly integrated with the helmet's other functionalities. The caregiver uses the app to set the pressure levels, select the audio tracks, and determine the specific tones that the child can access. This integration ensures that all manual controls are within safe and beneficial parameters.

Preset Pressure Levels: The caregiver configures the pressure levels that the air baffles will inflate to when the child presses the inflation button. These levels are based on data collected from previous sessions and are tailored to provide the most effective calming pressure for the child.

Audio Selections: The caregiver uploads and organizes the songs and tones available to the child through the manual audio control button. This ensures that the child only has access to sounds that have been proven to be beneficial.

Safety and Monitoring: The caregiver can monitor the use of the manual features through the app. Any manual activation by the child is logged, providing valuable data on how often and in what situations the child uses these features. This data can help refine the preset configurations for better effectiveness.

### Detailed Description—User-Friendly Design

The buttons are designed to be user-friendly and easy for the child to operate. They are strategically placed to minimize the risk of accidental activation and to ensure that the child can easily distinguish between the different functions.

Right Side Button: Clearly marked and positioned for inflating the air baffles.

Left Side Buttons: Two distinct buttons—one for releasing air pressure and one for controlling audio—placed close together but designed to prevent accidental inflation.

The manual activation feature of the therapeutic helmet provides a balanced approach to giving the child some control over their therapeutic environment while ensuring safety and effectiveness through caregiver oversight. By allowing the child to manually control the inflation of air baffles and access soothing audio, the helmet helps to empower the child and enhance their comfort during stressful situations. This feature, combined with the comprehensive control and monitoring capabilities of the caregiver app, ensures that the therapeutic benefits are maximized in a safe and controlled manner.

### Detailed Description—Patent Autistic Helmet Play List:

### Audio Features of the Therapeutic Helmet

The audio features of the therapeutic helmet are designed to provide calming and soothing auditory stimulation for children with autism. These features are highly customizable, allowing caregivers to select and upload specific sounds, music, and voice messages that are most effective for the child. Here's a detailed description of how these audio features work:

### Initial Sound Selection

Initially, the parent or caregiver determines the audio content based on their observations of how the child responds to various songs and tones. This initial selection is crucial in creating a baseline of sounds that are known to have a calming effect on the child. Over time, as more data is gathered, these selections can be refined to better suit the child's needs.

### Custom Voice Messages

The app allows parents to easily record and upload custom voice messages. By simply holding a button on their iPhone within the app, parents can record soothing and comforting messages. These messages are then automatically uploaded to the helmet and can be played back to the child when needed. This feature enables personalized communication that can provide immediate comfort and reassurance to the child.

### Uploading Songs and Tones

Caregivers can also upload songs and tones to the helmet through the app. Utilizing existing technologies that manage playlists on smartphones, the app provides a seamless way to integrate a variety of audio files into the helmet's system.

Playlist Integration: The app connects to the caregiver's existing music library on their iPhone. By selecting songs from this library, caregivers can create a customized playlist that is then uploaded to the helmet. The app supports various audio formats, ensuring compatibility with a wide range of music files.

Tone Selection: In addition to songs, caregivers can select specific tones known to have a calming effect. These tones can be white noise, nature sounds, or specific frequency tones that are therapeutic. The tones can be uploaded in the same manner as songs and added to the child's audio playlist.

### Real-Time Control and Playback

The audio features are designed for real-time control through the app. Caregivers can:

Play/Pause: Start or stop the audio playback at any time.

Skip Tracks: Move to the next song or tone in the playlist.

Adjust Volume: Control the volume of the audio playback to ensure it is at a comfortable and effective level for the child.

### Automatic and Manual Activation—the Audio Features can be Activated Both Automatically and Manually:

Automatic Activation: The app can be set to play specific audio tracks based on the child's pulse rate or other indicators of stress. For example, if an elevated pulse rate is detected, a soothing song or tone can automatically start playing.

Manual Activation: The child or caregiver can manually activate the audio features using the helmet's built-in controls. A button on the helmet allows the child to cycle through different songs and tones, giving them the autonomy to select the audio that they find most comforting.

### Detailed Description—Data-Driven Refinement

As the helmet is used, data on the child's responses to different audio tracks is collected and analyzed. This data helps in refining the audio selections to better meet the child's needs. For example, if a particular song consistently helps lower the child's pulse rate, it can be prioritized in the playlist.

Usage Logs: The app logs when and how often each audio track is played, along with the corresponding pulse rate data. This helps caregivers identify which sounds are most effective.

Feedback Loop: Based on the data collected, the app can suggest changes to the playlist, recommending tracks that have shown the most positive impact.

### Detailed Description—Seamless Integration

The audio system is seamlessly integrated with other helmet features, ensuring a holistic approach to managing the child's well-being. For instance, if the pulse rate monitoring system detects stress, it can trigger both the air baffles and the audio system simultaneously to provide a comprehensive calming effect.

Conclusion of Seamless Integration. The audio features of the therapeutic helmet provide a versatile and effective tool for calming and soothing children with autism. By leveraging modern technologies for playlist management and real-time data analysis, the system allows caregivers to customize and control the audio content in a way that is both responsive and adaptive to the child's needs. This personalized approach not only enhances the child's comfort but also contributes to a more effective overall therapeutic experience.

### Detailed Description—Autistic Helmet Sleep Monitoring

### Sleep Tracking Feature of the Therapeutic Helmet

The therapeutic helmet and wristband for children with autism is equipped with an advanced sleep tracking feature, designed to monitor sleep patterns including sleep duration and stages (light, deep, and REM sleep) and improve sleep quality through real-time interventions. The helmet uses a combination of sensors and pre-set data to detect the quality of the child's sleep and respond with soothing interventions. This comprehensive system ensures that children receive the best possible support to achieve restful and restorative sleep.

### Sleep Monitoring Sensors

The helmet is equipped with a variety of sensors to monitor the child's sleep patterns. These include:

Movement Sensors: Detects physical activity such as thrashing or restless movements during sleep. These sensors are strategically placed to capture even subtle movements, ensuring accurate monitoring.

Heart Rate Sensors: Monitors the child's pulse rate continuously. Changes in heart rate can indicate different sleep stages or potential disturbances.

Temperature Sensors: Measures the child's body temperature and ambient temperature inside the helmet. Temperature fluctuations can be indicative of discomfort that might disturb sleep.

### Data Collection and Analysis

The helmet collects data continuously throughout the night, providing a comprehensive overview of the child's sleep patterns. The types of data collected include:

Sleep Stages: Analysis of the different stages of sleep (light sleep, deep sleep, and REM sleep) based on movement and heart rate data. Understanding these stages helps in identifying disruptions and overall sleep quality.

Movement Patterns: Detailed logs of movements, identifying periods of restlessness or thrashing about.

Heart Rate Variability: Tracking changes in heart rate can provide insights into stress levels and sleep stages.

This data is processed in real-time by the helmet's onboard processor and transmitted wirelessly to the caregiver's iPhone app via Bluetooth. The app provides an interface for viewing and analyzing this data, allowing caregivers to understand the child's sleep patterns and identify any issues.

### Real-Time Interventions

When the helmet detects signs of restlessness or disturbances, it can automatically initiate soothing interventions based on pre-set data. These interventions are designed to help the child return to a restful state quickly:

Pressure Application: The helmet's air compression system can inflate the air baffles to apply gentle, calming pressure. This pressure is adjusted according to pre-set levels determined by the caregiver, based on what has been found to be effective for the child.

Soothing Sounds: The helmet can play calming sounds such as rain or white noise through its integrated speakers. These sounds can be selected and uploaded by the caregiver and are known to help the child relax and fall back asleep.

### Parental Alerts

In addition to automatic interventions, the helmet can send alerts to the parent's iPhone if significant disturbances are detected. These alerts ensure that the caregiver is aware of the child's condition and can intervene if necessary such as voice stress levels.

Movement Alerts: Notifications if the child is thrashing or showing significant restless movements.

Heart Rate Alerts: Notifications if the child's heart rate indicates stress or an unusual pattern.

Temperature Alerts: Notifications if the child's temperature is outside the comfortable range.

These alerts allow parents to take immediate action, such as adjusting the environment or manually triggering soothing features through the app.

### Customizable Settings

The caregiver app allows for extensive customization of the sleep tracking and intervention features. Caregivers can set:

Pressure Levels: Specific pressure levels for the air baffles during different types of disturbances.

Audio Selections: Upload and organize a playlist of soothing sounds and set rules for when each sound should be played.

Temperature Thresholds: Define comfortable temperature ranges and set automatic adjustments if the temperature goes outside these ranges.

Alert Sensitivity: Adjust the sensitivity of movement and heart rate alerts to ensure they are triggered appropriately without causing unnecessary interruptions.

These customizable settings ensure that the sleep tracking feature is tailored to the individual needs of the child, providing personalized and effective support.

### Data-Driven Improvements

The system continuously collects and logs sleep data, allowing for ongoing analysis and improvement of the intervention strategies. Over time, the app builds a detailed profile of the child's sleep patterns, identifying which interventions are most effective. This data-driven approach helps caregivers to refine the settings and improve the child's sleep quality progressively.

Sleep Reports: The app generates detailed sleep reports, providing insights into the child's sleep stages, disturbances, and overall sleep quality.

Trend Analysis: Identifies patterns and trends over time, helping caregivers understand the factors that influence the child's sleep.

Intervention Effectiveness: Evaluates the success of different interventions, allowing caregivers to optimize the settings for maximum benefit.

Integration with Other Features

The sleep tracking feature is seamlessly integrated with the helmet's other functionalities, providing a comprehensive solution for managing the child's well-being. For example, the pulse rate monitoring and real-time intervention systems work together to ensure that any disturbances during sleep are quickly addressed. The integration ensures that all data collected is used to provide the best possible support, whether the child is awake or asleep.

### Enhancing Sleep Quality

By leveraging advanced sensors and real-time data analysis, the helmet's sleep tracking feature offers a sophisticated and comprehensive approach to monitoring and improving the sleep quality of children with autism. By monitoring sleep patterns and responding to disturbances, the system provides personalized interventions that help manage sleep disturbances effectively. This contributes to better overall well-being, supporting both the child and the caregivers in achieving a more balanced and healthy lifestyle. The integration of customizable settings, parental alerts, and continuous data-driven improvements ensures that the helmet adapts to the child's needs, providing optimal support for restful and restorative sleep.

Restless Nights: For children who experience frequent restlessness or disturbances, the helmet's interventions can help maintain a more consistent sleep pattern.

Sleep Onset: The ability to play soothing sounds and apply calming pressure helps children fall asleep more easily.

Nighttime Comfort: Continuous monitoring of temperature and movement ensures that the child remains comfortable throughout the night, reducing the likelihood of waking up due to discomfort.

### Sleep Monitoring Conclusion

The sleep tracking feature of the therapeutic helmet offers a sophisticated and comprehensive approach to monitoring and improving the sleep quality of children with autism. By leveraging advanced sensors and real-time data analysis, the system provides personalized interventions that help manage sleep disturbances effectively. The integration of customizable settings, parental alerts, and continuous data-driven improvements ensures that the helmet adapts to the child's needs, providing optimal support for restful and restorative sleep.

### Detailed Description—Construction Materials

### Description—Autistic Helmet Materials Construction

### Construction and Materials of the Therapeutic Helmet

The therapeutic helmet for children with autism is designed with a focus on safety, comfort, and durability, incorporating several key components:

### Outer Shell

The outer shell of the helmet is made from a soft, semi-rigid material that provides protection while ensuring comfort. For children who may have more extreme behavioral episodes, a firmer plastic outer shell can be used. This firmer material offers enhanced protection against hard bumps and hits, ensuring the child's safety during severe mood swings. The design of the outer shell can be customized with various themes and colors to appeal to the child, such as an Iron Man theme for boys or a princess theme for girls.

### Inner Lining

The inner lining of the therapeutic helmet is crafted from a soft, breathable fabric that includes a velvet-like material for comfort. It is hypoallergenic and features moisture-wicking properties to keep the child's head dry and comfortable. The fabric is treated with antimicrobial agents to prevent the growth of bacteria. I Air Baffles and Compressor System

The helmet features air baffles made from durable, child-safe plastic, similar to those used in children's life vests and swimming pool rafts. The side of the baffles that contacts the child's head is coated with a soft, breathable fabric for additional comfort. A small air compressor, which can be a diaphragm-type, piston-driven, or rechargeable CO2 device, inflates the baffles to pre-set pressure levels determined by the caregiver. This pressure therapy helps calm the child during mood swings while ensuring quiet and efficient operation.

### Chin Strap

The helmet includes a comfortable chin strap to hold it securely in place. This strap is adjustable, allowing for a snug fit without causing discomfort. It is padded with soft, breathable material to prevent irritation, ensuring the child can wear the helmet comfortably for extended periods.

### Therapeutic Helmet's Construction Conclusion

The therapeutic helmet's construction and materials are carefully chosen to provide a balance of safety, comfort, and durability. The outer shell, inner lining, air baffles, compressor system, and chin strap all work together to create a supportive and protective environment for children with autism.

### Detailed Description Autistic Helmet Additional Features:

A central database that can evaluate the data for the benefit of all of the children. A monthly charge to connect to the database.

### Proposal for Data-Driven Subscription Service for Therapeutic Helmet Users

As part of the therapeutic helmet's comprehensive solution for managing severe mood swings in children with autism, there is potential to enhance its functionality and support through a data-driven subscription service. This service would involve collecting and analyzing data from helmets used by children across the country. By aggregating this data, the service could identify the most effective therapeutic interventions based on specific physiological indicators, such as pulse rate, body temperature, and movement patterns.

The primary objective of this subscription service would be to continuously refine and optimize the therapeutic protocols employed by the helmets. By evaluating data on a national scale, the service could determine which soothing tones, songs, or pressure levels are most effective in calming children during mood swings. For instance, if a significant number of children exhibit a lower pulse rate and reduced agitation when exposed to a particular type of music or pressure level, this information could be used to update and customize the therapeutic settings for all users.

The Process would Involve Several Key Steps:

Data Collection: Each therapeutic helmet would securely transmit anonymized data to a central database. This data would include real-time readings of pulse rate, temperature, movement, and the specific interventions applied (e.g., pressure adjustments, audio playback).

Data Analysis: Advanced analytics and machine learning algorithms would process this data to identify patterns and correlations. The analysis would focus on determining the most effective interventions based on various physiological indicators. For example, the system could analyze whether a specific song consistently helps reduce the pulse rate during high-stress moments or if a particular pressure setting is more effective in calming physical agitation.

Recommendations and Updates: Based on the analysis, the service would provide personalized recommendations to caregivers through the remote control app. These recommendations would include optimized settings for pressure therapy, audio selections, and other interventions. Additionally, the service could periodically update the helmet's firmware to incorporate new findings and ensure that all users benefit from the latest insights.

Continuous Improvement: The subscription service would operate on a continuous feedback loop, where data collected from ongoing use is regularly analyzed to refine and improve therapeutic protocols. This ensures that the helmet's interventions remain effective and adaptive to the evolving needs of each child.

The subscription service could offer several tiers, with basic and premium options. The basic tier might include regular updates and general recommendations, while the premium tier could offer more personalized analysis, one-on-one consultations with specialists, and access to exclusive content such as new therapeutic audio tracks and advanced monitoring features.

This data-driven approach not only enhances the therapeutic efficacy of the helmets but also provides invaluable insights into the treatment of autism-related mood swings on a broader scale. By leveraging large-scale data analysis, the service could contribute to the overall understanding of autism and the development of new therapeutic techniques.

Considering the significant potential benefits, this subscription service should be included in the patent process as an integral component of the therapeutic helmet system. Protecting this innovative aspect through the patent would secure the commercial viability of the service and ensure that it remains a proprietary advantage of the product.

By offering this subscription service, the therapeutic helmet can provide continuous, data-driven support to children with autism and their caregivers, ensuring the most effective and personalized care possible. This innovative approach not only sets the helmet apart from existing solutions but also underscores its commitment to leveraging technology for improved health outcomes.

### Detailed Description—Alternate Helmet Material

### Examples of Helmet Materials

### Soft and Pliable Helmets

### Thermoplastic Polyurethane (TPU)

Properties: TPU is flexible, elastic, and offers good impact resistance. It is soft to the touch and can adapt to different shapes, providing comfort without full head contact.

Advantages: Ensures a comfortable fit and absorbs minor impacts. The pliability of TPU helps accommodate the inflatable baffles without causing discomfort to the child.

Use Case: Suitable for children with mild to moderate symptoms who need a soft, flexible helmet that provides comfort and safety.

### Silicone Rubber

Properties: Silicone rubber is highly flexible, durable, and hypoallergenic. It is soft and gentle on the skin, making it ideal for prolonged contact.

Advantages: Provides excellent cushioning and can conform to various head shapes. Its softness and flexibility complement the inflatable baffles, ensuring the child remains comfortable even during inflation.

Use Case: Ideal for younger children or those who require a soft and adaptive helmet to ensure maximum comfort.

### Impact-Resistant and Durable Helmets

### Polycarbonate (PC)

Properties: Polycarbonate is a strong, impact-resistant plastic that is lightweight and durable. It can withstand significant impacts, providing robust protection.

Advantages: Offers high levels of protection against hard impacts while being relatively lightweight. Its durability ensures that the helmet remains effective over time, even in severe cases.

Use Case: Suitable for children with severe symptoms who require a helmet capable of withstanding significant impacts.

### High-Density Polyethylene (HDPE)

Properties: HDPE is a hard, impact-resistant plastic known for its toughness and durability. It is resistant to chemicals and UV radiation, making it suitable for various environments.

Advantages: Provides excellent impact resistance and durability, ensuring the helmet can protect against severe impacts. Its rigidity complements the protective needs of children with more intense behaviors.

Use Case: Best for children with severe mood swings or self-harming behaviors who need a durable and protective helmet.

### Summary of Material Choices

These material choices for the therapeutic helmet ensure that it can provide both comfort and protection tailored to the child's needs. Soft and pliable materials like TPU and silicone rubber offer flexibility and comfort, while impact-resistant materials like polycarbonate and HDPE provide the necessary protection for more severe cases. The integration of inflatable baffles further enhances the helmet's ability to deliver therapeutic benefits without compromising on safety or comfort.

Detailed Description—More Details about the Function and Control of the Pressure Areas

### Detailed Description of the Pressure Therapy Baffles in the Therapeutic Helmet

The therapeutic helmet for children with autism features advanced pressure therapy baffles designed to apply calming pressure in various patterns and rhythms. These baffles, integrated into the helmet, can inflate and deflate in controlled cycles, providing a dynamic and customizable therapeutic experience.

### Pressure Patterns and Rhythms

### Cyclic Pressure Application:

The baffles can inflate to a pre-set pressure level and then gradually deflate, creating a rhythmic pressure cycle. This cycle can be adjusted to control how quickly the pressure builds and dissipates, as well as the duration of each interval between pressure applications.

The baffles can inflate to a pre-set pressure level and then gradually deflate, creating a rhythmic pressure cycle. This cycle can be adjusted to control how quickly the pressure builds and dissipates, as well as the duration of each interval between pressure applications.

Example: Light pressure begins to build to a set level, holds momentarily, and then slowly dissipates. This cycle can repeat at controlled intervals, providing a soothing rhythm that helps calm the child.

### Targeted Pressure Areas:

The baffles can target specific areas of the head, applying pressure in a localized manner before spreading to other regions. This allows for a more focused therapeutic effect where needed.

Example: Pressure starts at the base of the neck, gradually encompassing the neck and rising to cover the entire head. The deflation phase then follows in the reverse order, ensuring a gradual release of pressure.

### Pulse and Wave Pressure:

The helmet can create pulsing pressure patterns where the baffles inflate and deflate in rapid succession or slow waves. These patterns can be adjusted based on the child's response and therapeutic needs.

Example: Pressure is applied in pulses, where the baffles quickly inflate and deflate at set intervals, or in a wave pattern, where pressure moves from one side of the head to the other in a gentle, rolling motion.

### Zone-Specific Pressure Application:

The baffles can inflate in specific zones, such as the top, sides, or back of the head, independently of each other. This allows for customized pressure applications that can cater to different calming needs.

Example: Pressure is applied only to the sides of the head or the top of the head, depending on where the child benefits most from the pressure.

### Customizable Configurations

The helmet's pressure therapy system is highly configurable, allowing caregivers to set various parameters based on the child's unique needs. These configurations include:

Pressure Levels: Adjusting the amount of pressure applied during inflation.

Inflation and Deflation Speed: Controlling how quickly the baffles inflate to the desired pressure and how slowly they deflate.

Interval Timing: Setting the duration between pressure cycles, allowing for continuous or intermittent pressure application.

Pattern Selection: Choosing from different inflation patterns (cyclic, pulsing, wave, or zone-specific).

Integration with Monitoring Systems

The pressure therapy system is integrated with the helmet's monitoring systems, which track the child's physiological indicators such as heart rate, body temperature, and movement. This integration allows the system to adjust the pressure patterns in real-time based on the child's current state, ensuring optimal therapeutic effectiveness.

Real-Time Adjustments: The helmet can modify pressure cycles dynamically in response to sensor data, such as increasing pressure when a high heart rate is detected to provide additional calming.

Personalized Therapy: Data collected from the helmet over time helps refine the pressure therapy settings, creating a personalized profile that maximizes the therapeutic benefits for the child.

The therapeutic helmet's pressure therapy system offers a sophisticated approach to calming children with autism through customizable pressure patterns. By controlling the inflation and deflation of baffles in various rhythms and targeting specific areas of the head, the helmet provides a dynamic and adaptable therapeutic experience. This system, integrated with real-time monitoring and data-driven adjustments, ensures that each child receives personalized and effective pressure therapy tailored to their unique needs.

### DESCRIPTION OF THE DRAWING

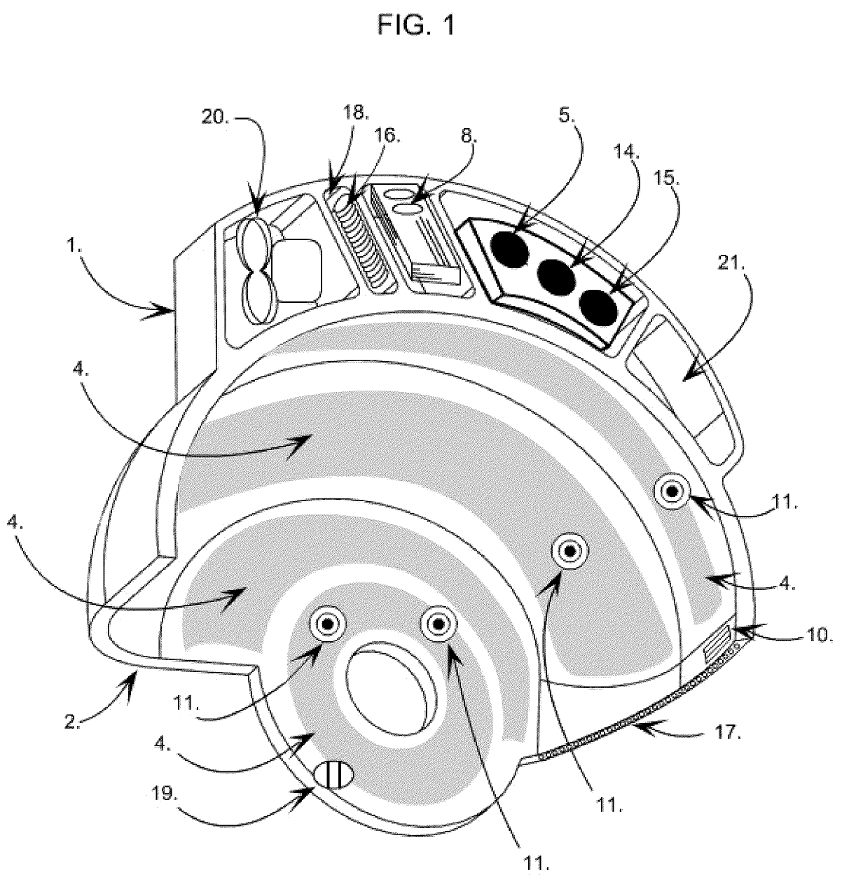

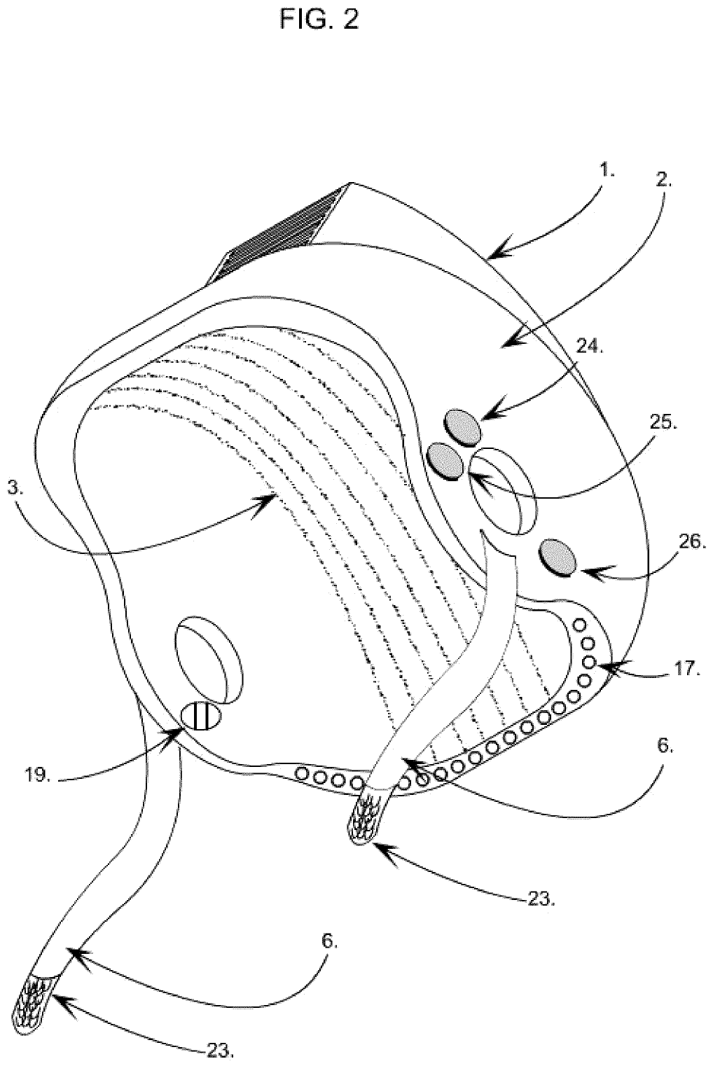

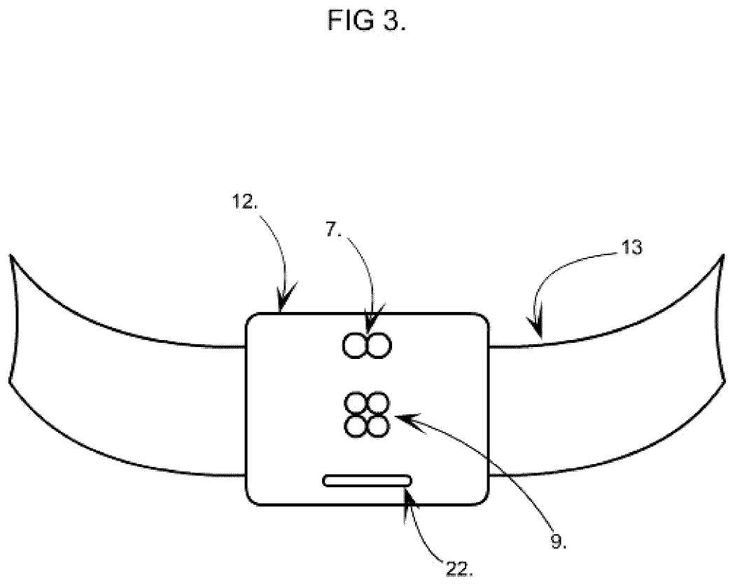

Turning now descriptively to the drawings, in which similar reference in characters denote the similar elements throughout the several views, the figures illustrate a Customizable Pressure Therapy Autism Helmet With regard to the reference numerals used, the following numbering is used throughout the various drawing's figures:
FIG. 1
1. Helmet
2. Outer shell
4. Air baffles
5. Small air compressor
8. Bluetooth
10. Motion sensors (helmet)
11. Speakers
14. Manual pump
15. Rechargeable CO2 device
16. Battery pack
17. Ventilation system
18. Battery compartment
19. Microphone
20. Cooling Fan
21. Water Reservoir Compartment
FIG. 2.
1. Helmet
2. Outer shell
3. Inner lining
6. Comfortable chin strap
17. Ventilation system
19. Microphone
23. Quick Release chin strap
24. Manual pressure control
25. Air pressure release button
26. Audio control button
FIG. 3.
7. Heart rate monitor
9. Motion sensor (wristband)
12. Wristband device
13. Quick release wristband
22. Temperature sensor

### Description of the Referenced Number

1. Helmet: The main structure that houses all components.
2. Outer shell: The exterior part of the helmet provides protection.
3. Inner lining: The interior part of the helmet is made from soft, breathable fabric.
4. Air baffles: Inflatable components that provide adjustable pressure therapy.
5. Small air compressor: A device for inflating the air baffles.
6. Comfortable chin strap: A strap designed for securing the helmet comfortably on the child's head.
7. Heart rate sensors: Sensors that measure the child's pulse rate using light-based technology.
8. Bluetooth: Wireless technology for data transmission between the helmet and the app.
9. Motion sensors: Sensors that detect and record the child's physical activity and restlessness. (wristband)
10. Motion sensors: Sensors that detect and record the child's physical activity and restlessness. (helmet)
11. Speakers: Audio output devices embedded in the helmet for playing sounds.
Pre-recorded voice messages: Customizable audio messages recorded by caregivers.
Soothing tones: Calming sounds played through the helmet's audio system.
Music: Tracks selected by the caregiver to soothe the child.
12. Wristband: a wearable device equipped with sensors for monitoring motion, temperature, and heart rate, providing continuous physiological data to the therapeutic helmet system.
13. Quick release wristband: a wearable device featuring a quick-release mechanism for easy removal.
14. Manual pump: A hand-operated device for inflating the air baffles.
15. Rechargeable CO2 device: A component for rapid inflation of the air baffles using compressed CO2.
16. Battery pack: A power source for the helmet's electronic components.
17. Ventilation system: Components that ensure proper airflow and prevent overheating.
18. Battery compartment: A dedicated enclosure located at the front top of the helmet designed to house and secure the battery pack. This compartment ensures easy access for battery replacement or recharging and is integrated with the helmet's electronics to power various systems.
19. Microphone: A sensitive audio input device located near the child's mouth inside the helmet. Its placement ensures the accurate capture of the child's vocalizations, allowing for precise monitoring of stress, emotions, happiness, and non-stress states. The data collected by the microphone system is transmitted to the helmet's processor for real-time analysis and intervention.
20. Cooling fan: works in conjunction with the water reservoir and the ventilation system to regulate temperature.
21. Water reservoir: functions with the cooling fan and ventilation system to regulate temperature.
22. Temperature sensors: Sensors that measure the ambient and body temperature inside the helmet.
23. Quick release chin strap: a wearable chin strap device featuring a quick-release mechanism for easy removal.
24. A button on the left side of the helmet allows the child to manually control the inflation of the air baffles to a pre-set pressure level determined by the caregiver.
25. A button on the left side of the helmet, located below the inflation button, that allows manually deflating the air baffles.
26. Adjacent to the air pressure release button on the left side, another button controls the helmet's audio features, cycling through pre-set audio options such as favorite songs and soothing tones.

### Preferred Embodiment

The preferred embodiment of the therapeutic helmet for children with autism comprises an outer shell made from a material with a Shore A hardness of 60-80, such as thermoplastic polyurethane (TPU), or a firmer plastic such as acrylonitrile butadiene styrene (ABS), designed to provide enhanced head injury protection. The inner lining is crafted from soft, breathable fabric including a velvet-like material, ensuring comfort and preventing overheating. The helmet incorporates air baffles made from durable, child-safe plastic with a soft coating, which are inflated by a small air compressor to pre-set pressure levels determined by the caregiver, providing customizable pressure therapy to calm the child during mood swings. Integrated into the helmet are advanced monitoring systems, including heart rate sensors, temperature sensors, and movement sensors, as well as two strategically placed microphones near the child's mouth to detect verbal stress. A wristband worn by the child also collects vital physiological data such as heart rate, body temperature, and movement, continuously transmitting this data to the helmet for a comprehensive assessment of the child's physiological state. The helmet further features an integrated audio system with speakers embedded within the inner lining, capable of playing pre-recorded voice messages, soothing tones, and music, with functionalities for real-time audio adjustments via a remote control app. The outer shell can be customized with various themes and colors to appeal to children, and includes impact-absorbing materials for additional protection. The helmet's chin strap is adjustable, includes a quick-release mechanism for emergencies, and is made of durable nylon webbing. The air compressor can be a rechargeable CO2 device, supplemented by a manual pump as an alternative inflation method. Additionally, the helmet offers a sleep tracking feature, continuously monitoring sleep quality and initiating preemptive actions to help the child sleep better and longer. The integration of these advanced features and technologies ensures that the therapeutic helmet provides early detection, proactive intervention, and continuous support, optimizing therapeutic interventions and ensuring the child's comfort and safety.

What is claimed is:

1: The therapeutic helmet for children with autism, comprising:
an outer shell made from a material with a Shore A hardness of 60-80, such as thermoplastic polyurethane (TPU), or a firmer plastic such as acrylonitrile butadiene styrene (ABS), designed to provide enhanced head injury protection;
an inner lining crafted from soft, breathable fabric including a velvet-like material; air baffles made from durable, child-safe plastic with a soft coating;
a small air compressor for inflating the air baffles to pre-set pressure levels; two strategically placed microphones on each side near the mouth of the child for detecting verbal stress;
and a comfortable, adjustable chin strap to hold the helmet in place.

2: The therapeutic helmet of claim 1, wherein the inner lining further comprises integrated monitoring systems, including:
heart rate sensors designed to detect and monitor the child's pulse rate, temperature sensors configured to measure the child's body temperature,
movement sensors capable of detecting and recording the child's physical activity levels,
a wristband worn by the child, equipped with sensors to collect vital physiological data, such as heart rate, body temperature, and movement;
wherein these sensors are embedded within the inner lining and connected to a control module that communicates wirelessly with a remote control application on a mobile device, enabling the monitoring and analysis of the child's physiological and activity data, and triggering pre-set therapeutic interventions based on predefined criteria.

3: The therapeutic helmet of claim 1, further comprising an integrated audio system with speakers, wherein the audio system includes:
speakers embedded within the inner lining, positioned near the child's ears for optimal sound delivery and quality, designed to deliver 3D surround sound for an immersive audio experience;
digital audio player capable of storing and playing pre-recorded voice messages, soothing tones, and music;
a wireless communication module enabling the caregiver to select and upload audio content to the digital audio player through a remote control application on a mobile device;
a playlist feature that allows the caregiver to organize and select specific tracks; a noise-cancellation feature to block out external sounds;
support for multiple languages to provide customized user experiences;
a sleep mode that automatically plays relaxing music or white noise to help the child fall asleep;
functionalities within the remote control app allowing real-time audio adjustments based on the child's current state;
the capability for the caregiver to record and upload custom voice messages; control over the audio system's volume and track selection by the child through buttons on the helmet;
the ability to be triggered automatically by sensor data to play soothing sounds during moments of distress.

4: The therapeutic helmet of claim 1, wherein the outer shell is designed with customizable themes and colors, including popular characters and motifs to appeal to children, and is constructed from impact-absorbing materials to further protect against bumps and hits.

5: The therapeutic helmet of claim 1, wherein the air baffles are configured to apply adjustable pressure levels controlled via a remote control application based on pre-set therapeutic profiles, and are arranged in a pattern to evenly distribute pressure across the child's head.

6: The therapeutic helmet of claim 1, wherein the chin strap includes a quick-release mechanism for easy removal in case of emergency, and is made of durable nylon webbing.

7: The therapeutic helmet of claim 1, wherein the inner lining includes antimicrobial properties to prevent the growth of bacteria and ensure hygiene, and is made of moisture-wicking fabric to keep the child's head dry and comfortable.

8: The therapeutic helmet of claim 1, wherein the air compressor is a rechargeable CO2 device for rapid inflation and deflation of the air baffles, and further comprising a manual pump as an alternative method for inflating the air baffles.

9: The therapeutic helmet of claim 2, wherein the heart rate sensors accurately detect and monitor pulse rate using advanced heart rate monitoring technology, and the temperature sensors continuously monitor the ambient temperature inside the helmet to ensure comfort.

10: The therapeutic helmet of claim 2, wherein the movement sensors detect patterns of restlessness or thrashing during sleep, and are calibrated to differentiate between different types of physical activities.

11: The therapeutic helmet of claim 2, further comprising an alert system that notifies the caregiver via the remote control app if abnormal readings are detected, and the remote control app logs all sensor data for later review and analysis by the caregiver.

12: The therapeutic helmet of claim 2, wherein the remote control app can adjust therapeutic interventions based on real-time sensor data, and the heart rate monitoring system includes a feedback mechanism to adjust pressure levels in response to stress indicators.

13: The therapeutic helmet of claim 2, wherein the remote control app provides visual and auditory feedback to the child based on the sensor data, and the temperature sensors trigger cooling or heating elements within the helmet to maintain an optimal temperature.

14: The therapeutic helmet of claim 1, further comprising a sleep tracking feature that monitors sleep quality, including sleep duration and stages (light, deep, and REM sleep), providing a comprehensive assessment of the child's sleep patterns and initiating preemptive actions to help the child sleep better and longer.

15: The therapeutic helmet of claim 2, wherein the wristband continuously transmits the collected data to the helmet, allowing for a comprehensive assessment of the child's physiological state, and the integration of the auditory data from the microphones with the physiological data from the wristband provides a holistic approach to monitoring the child's well-being accurately and providing timely interventions.

16: The therapeutic helmet of claim 1, further comprising a system for creating and storing a personalized therapeutic profile for the child based on the collected data, which is used to optimize the therapeutic interventions and ensure the child's comfort and safety.
